(19) United States
(12) Patent Application Publication
(10) Pub. No.: US 2023/0326556 A1
(43) Pub. Date: Oct. 12, 2023

(54) SYSTEMS AND METHODS FOR PERFORMING ADDITIVE SMOOTHING ON LOW-COVERAGE SEQUENCING DATA FROM A NUCLEIC ACID SAMPLE

(71) Applicant: GRAIL, LLC, Menlo Park, CA (US)

(72) Inventors: Robert Abe Paine CALEF, Redwood City, CA (US); Eric Michael SCOTT, Redwood City, CA (US); Karina SAMUEL-GAMA, Long Beach, CA (US)

(73) Assignee: GRAIL, LLC, Menlo Park, CA (US)

(21) Appl. No.: 18/194,250

(22) Filed: Mar. 31, 2023

Related U.S. Application Data
(60) Provisional application No. 63/362,667, filed on Apr. 8, 2022.

(51) Int. Cl. G16B 30/00; G16B 40/20; G16H 50/50

(52) U.S. Cl. CPC G16B 30/00; G16H 50/50; G16B 40/20

(57) ABSTRACT

Systems and methods for reducing noise for the analysis of low coverage sequencing data from a nucleic acid sample using a method, including: receiving, at an input component of the system, a set of sequence reads associated with the nucleic acid sample; allocating, using a processor component of the system, the set of sequence reads into a plurality of genomic bins; and introducing, subsequent to the allocating, a pseudocount number to bincount values to produce a smoothed dataset, wherein each of the bincount values is associated with one of the plurality of genomic bins. Other aspects are described and claimed.

### CROSS REFERENCE TO RELATED APPLICATIONS

This application claims priority to U.S. Provisional Application No. 63/362,667, filed on Apr. 8, 2022, which is incorporated by reference herein in its entirety.

### TECHNICAL FIELD

The present disclosure relates generally to the field of computational data analysis and, more particularly, to systems and methods of reducing noise for the analysis of low coverage sequencing data from a nucleic acid sample.

### BACKGROUND

Advances in research and technology have led to the development of new techniques for detecting various disease states, such as cancer, at an earlier stage. For instance, the isolation, sequencing, and analysis of cell-free DNA (“cfDNA”) fragments from blood samples has been shown to be an effective method for calling cancer signals, i.e., the cancer signal of original (“CSO”), and for identifying the tissue from which the cancer originates, i.e., the tissue of origin (“TOO”). Although these techniques work well with higher coverage whole genome sequencing (“WGS”) data (e.g., sequencing conducted at 30× coverage), issues may arise in resultant datasets when the sequencing depth is much lower (e.g., at 1× coverage). The present disclosure is accordingly directed to improving the quality of low coverage sequencing data by utilizing an additive smoothing technique.

The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

In summary, one aspect provides a method of reducing noise for the analysis of low coverage sequencing data from a nucleic acid sample using a system, the method including: receiving, at an input component of the system, a set of sequence reads associated with the nucleic acid sample; allocating, using a processor component of the system, the set of sequence reads into a plurality of genomic bins; and introducing, subsequent to the allocating, a pseudocount number to bincount values to produce a smoothed dataset, wherein each of the bincount values is associated with one of the plurality of genomic bins.

Another aspect provides a system for reducing noise for the analysis of low coverage sequencing data from a nucleic acid sample, the system including: a database; and at least one processing component configured to perform operations including: receiving a set of sequence reads associated with the nucleic acid sample; allocating the set of sequence reads into a plurality of genomic bins; and introducing, subsequent to the allocating, a pseudocount number to bincount values to produce a smoothed dataset, wherein each of the bincount values is associated with one of the plurality of genomic bins.

A further aspect provides a non-transitory computer-readable medium storing instructions for reducing noise for the analysis of low coverage sequencing data from a nucleic acid sample, the instructions, when executed by one or more processors, causing the one or more processors to perform operations including: receiving a set of sequence reads associated with the nucleic acid sample; allocating the set of sequence reads into a plurality of genomic bins; and introducing, subsequent to the allocating, a pseudocount number to bincount values to produce a smoothed dataset, wherein each of the bincount values is associated with one of the plurality of genomic bins.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass±10% of a specified amount or value. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein, the terms “comprises,” “comprising,” “including,” “having,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” In addition, the term “between” used in describing ranges of values is intended to include the minimum and maximum values described herein.

### BRIEF DESCRIPTION OF THE DRAWINGS

The accompanying drawings, which are incorporated in and constitute a part of this specification, illustrate several embodiments and together with the description, serve to explain the principles of the disclosure.

FIG. 1A depicts an exemplary computer system for executing the techniques described herein.

FIG. 1B depicts an exemplary software platform for executing the techniques described herein.

FIG. 2A illustrates a flowchart of an exemplary method of reducing noise for the analysis of low coverage sequencing data, according to one aspect of the present disclosure.

FIG. 2B illustrates a flowchart of another exemplary method of reducing noise for the analysis of low coverage sequencing data, according to one aspect of the present disclosure.

FIG. 3A depicts a graphical representation of results associated with an original set of sequence reads, according to one aspect of the present disclosure.

FIG. 3B depicts a graphical representation of results associated with a pseudocount-modified set of sequence reads, according to one aspect of the present disclosure.

FIG. 4A depicts a graphical representation of the limit of detection (LoD) for original off-target data associated with a targeted DNA methylation assay, according to one aspect of the present disclosure.

FIG. 4B depicts a graphical representation of the limit of detection (LoD) for pseudocount-modified off-target data associated with a targeted DNA methylation assay, according to one aspect of the present disclosure.

### DETAILED DESCRIPTION OF EMBODIMENTS

The following embodiments describe systems and methods for improving the quality of low coverage sequencing data from a nucleic acid sample. More particularly, the embodiments contemplated in the present disclosure may reduce the noise metrics found in low coverage sequencing data through the addition of a pseudocount number to bincount values associated with each genomic bin in a processed set of sequence reads.

DNA methylation has long been regarded as a hallmark of cancer and holds great promise for early-stage cancer detection. In particular, through the utilization of targeted whole genome bisulfite sequencing (“WGBS”), coupled with the processing capabilities associated with machine learning technologies, methylated DNA sequences can be effectively read and abnormally methylated sequences (i.e., those that may be indicative of cancer) may be identified. Accordingly, targeted DNA methylation assays performed on cell-free DNA (“cfDNA”) fragments may be capable of detecting multiple cancers across all stages, including at early stages when treatment may be more effective.

Situations have arisen where a cancer signal may be detected using a particular assay (e.g., a targeted DNA methylation assay, a somatic copy number alteration (SCNA) assay, etc.) but a corresponding tumor is not found by a clinician. In these situations, the detected cancer signal may be initially disregarded and/or categorized as a “false positive.” In an effort to provide orthogonal evidence for these detected cancer signals, one or more alternative assays may be conducted based on the same cfDNA sample. For example, in a situation where a cancer signal is derived from a targeted DNA methylation assay, an alternative assay may be conducted on the off-target data associated with the targeted DNA methylation assay (i.e., on the portions of the genome not covered by the panel regions in the targeted DNA methylation assay) to determine whether additional indications of cancer are present.

One viable alternative assay, for example, may be an SCNA assay, which may be used to identify the presence or absence of specific copy number alterations known to be prevalent in different types of cancer. During a conventional implementation of an SCNA assay (i.e., on a primary dataset), one step is the normalization of the sequence reads that fall in a particular genomic region so that the bincounts will be comparable across samples. The normalization process may include multiple normalization steps, a first of which may correspond to within-sample normalization of the sequence reads based on the median bincount within a sample. Other normalization steps (e.g., involving cross-sample normalization with a set of “baseline” samples, etc.) may thereafter occur.

Generally, this within-sample median normalization has been found to produce reliable results at higher coverage WGS (e.g., having 30× depth of coverage) but does not work as well when utilized in conjunction with lower coverage sequence reads (e.g., having <1× depth of coverage). More particularly, these low coverage samples typically have an extremely low median bincount, which may translate to a noisy signal that has a negative effect on classification performance (i.e., the noisy signal may generate a false positive cancer call). Due to the fact that calling SCNAs from the off-target DNA methylation data may be more-or-less equivalent to calling SCNAs from low coverage WGS data, similar issues may arise during attempts to validate the original cancer call of the DNA methylation assay.

Solutions currently exist for removing noise in low depth samples to limit or prevent spurious cancer calls. For instance, certain samples (e.g., those with median bin depths below 10) may be excluded during cross-sample normalization. Although such a process may ultimately reduce the noise in a dataset, the exclusion of entire samples on the basis of their coverage may risk excluding potentially important sequencing information. As another example, the existing training data for a classification model may be subsampled to a similar depth of distribution as the low coverage sample. Such a process, however, may be time-consuming, costly, remove useful data, and/or burdensome. Accordingly, a solution is needed that is capable of effectively reducing the noise generated during the analysis of low coverage sequence reads while still preserving the available sequencing data.

To address the above-noted problems, the present disclosure describes an approach for smoothing out the categorical data produced from low coverage sequencing so that it can be effectively utilized in one or more downstream applications. More particularly, an additive, or “Laplace”, smoothing process may be applied to a low coverage dataset in which a constant value (i.e., a “pseudocount”) is added to each genomic bin prior to normalization. In practice, such a process may reduce some of the noise in the low coverage samples, which may improve classification performance and/or the limit of detection (LoD) of the classification model.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” as used herein does not necessarily refer to a different embodiment. It is intended, for example, that claimed subject matter include combinations of exemplary embodiments in whole or in part.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

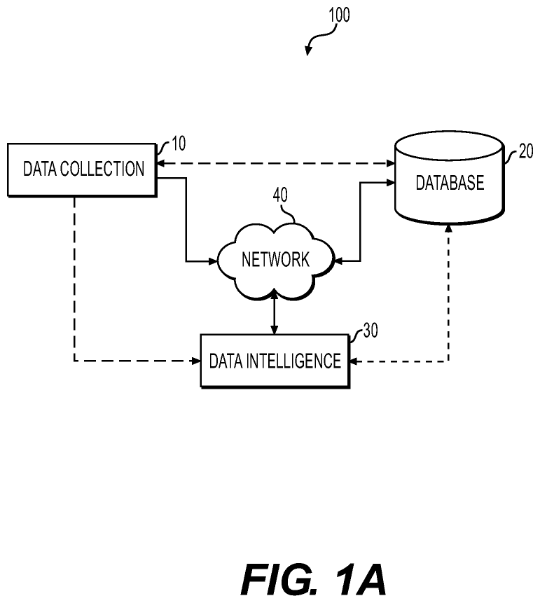

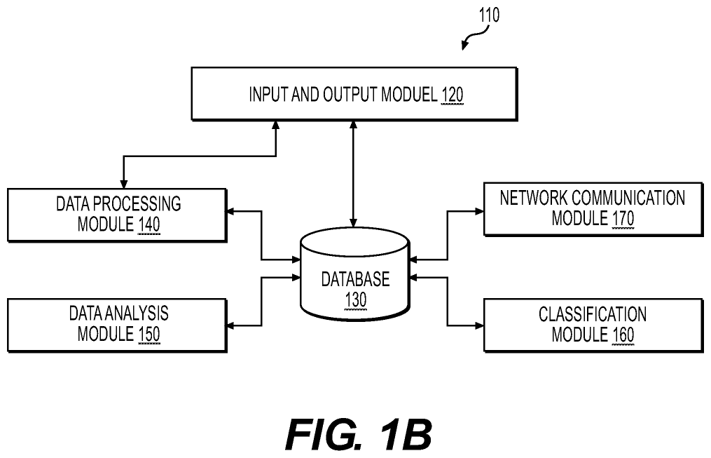

FIG. 1A depicts an exemplary system for processing low coverage sequencing data. Exemplary system 100 includes a data collection component 10, a database 20 and device data intelligence component 30, connected to each other via network 40. Alternatively, or additionally, one or more of the components may be connected with another component locally without reliance on network connection; e.g., through a wired connection. Sequencing data of cell-free nucleic acids are used to illustrate the concepts. However, one of skill in the art would understand that the current method may be applied to sequencing data of other materials or non-sequencing data as well.

As disclosed herein, data collection component 10 may include a device or machine with which sequencing data may be generated. In some embodiments, data collection component 10 may include a sequencing machine or a facility that uses a sequencing machine to generate nucleic acid sequence data of biological samples. Any applicable biological samples may be used. In some embodiments, a biological sample is cell-based; for example, one or more types of tissue. In some embodiments, a biological sample is a sample that includes cell-free nucleic acid fragments. Examples of biological samples include, but are not limited to, a blood sample, a serum sample, a plasma sample, a urine sample, a saliva sample, and etc.

Examples of sequencing data may include, but are not limited to, sequence read data of targeted genomic locations, partial or whole genome sequencing data of genome represented by nucleic acid fragments in cell-free or cell-based samples, partial or whole genome sequencing data including one or more types of epigenetic modifications (e.g., methylation), or combinations thereof.

Data acquired by the data collection component 10 may be transferred to database 20 via network 40. In some embodiments, the collected data may be analyzed by data intelligence component 30, via local or network connection. FIG. 1B depicts exemplary functional modules that may be implemented to perform tasks of data intelligence component 30.

FIG. 1B depicts an exemplary computer system 110 for processing sequencing data (e.g., low coverage sequencing data) and introducing pseudocounts thereto to reduce the resultant noise. Exemplary embodiment 110 achieves such functionalities by implementing, on one or more computer devices, user input and output (I/O) module 120, memory or database 130, data processing module 140, data analysis module 150, classification module 160, network communication module 170, and any other functional modules that may be needed for carrying out a particular task (e.g., an error correction or compensation module, a data compression module, and etc.). As disclosed herein, user I/O module 120 may further include an input sub-module, such as a keyboard, and an output sub-module, such as a display (e.g., a printer, a monitor, or a touchpad). In some embodiments, all functionalities are performed by one computer system. In some embodiments, the functionalities are performed by more than one computer.

Also disclosed herein, a particular task may be performed by implementing one or more functional modules. In particular, each of the enumerated modules itself may, in turn, include multiple sub-modules. For example, data processing module 140 may include a sub-module for data quality evaluation (e.g., for discarding very short sequence reads or sequence reads including obvious errors), a sub-module for normalizing numbers of sequence reads that align to different regions of a reference genome, a sub-module to compensate/correct GC biases, and etc.

In some embodiments, a user may use I/O module 120 to manipulate data that is available either on a local device or can be obtained via a network connection from a remote service device or another user device. For example, I/O module 120 may allow a user, e.g., via a keyboard, a mouse, or a touchpad, to perform data analysis via a graphical user interface (GUI). In some embodiments, a user may manipulate data via voice control. In some embodiments, user authentication may be required before a user is granted access to the data being requested.

In some embodiments, user I/O module 120 may be used to manage various functional modules. For example, a user may request via user I/O module 120 input data while an existing data processing session is in process. A user may do so by selecting a menu option or type in a command discretely without interrupting the existing process.

As disclosed herein, a user may use any type of input to direct and control data processing and analysis via I/O module 120.

In some embodiments, system 110 further comprises a memory or database 130. In some embodiments, database 130 comprises a local database that may be accessed via user I/O module 120. In some embodiments, database 130 comprises a remote database that may be accessed by user I/O module 120 via network connection. In some embodiments, database 130 is a local database that stores data retrieved from another device (e.g., a user device or a server). In some embodiments, memory or database 130 may store data retrieved in real-time from internet searches.

In some embodiments, database 130 may send data to and receive data from one or more of the other functional modules, including, but not limited to, a data collection module (not shown), data processing module 140, data analysis module 150, classification module 160, network communication module 170, and etc.

In some embodiments, database 130 may be a database local to the other functional modules. In some embodiments, database 130 may be a remote database that may be accessed by the other functional modules via wired or wireless network connection (e.g., via network communication module 170). In some embodiments, database 130 may include a local portion and a remote portion.

In some embodiments, system 110 comprises a data processing module 140. Data processing module 140 may receive the real-time data, from I/O module 120 or database 130. In some embodiments, data processing module 140 may perform standard data processing algorithms such as one or more of noise reduction, signal enhancement, normalization of counts of sequence reads, correction of GC bias, and etc. In some embodiments, data processing module 140 may identify global or local systematic errors. For example, sequencing data may be aligned to regions within a reference genome. The numbers of sequence reads aligned to different genomic regions may vary for the same subject. The numbers of sequence reads aligned to the same genomic regions may vary between subjects. Some of these differences, especially those observed in healthy subjects, may result from systematic errors instead of an association with one or more diseased conditions. For example, if sequencing data corresponding to a particular genomic region shows wide ranges of variation between healthy subjects, data processing module 140 may classify the particular genomic region as a high-noise region and may exclude the corresponding data from further analysis. In some embodiments, instead of exclusion, data processing module 140 may attempt to reduce the noise in a resultant dataset by, for example, smoothing out the dataset via introduction of a predetermined pseudocount number to each genomic bin in a set of sequence reads, as further described herein. In some embodiments, the identification and treatment of possible systematic errors may be performed by data analysis module 140, as illustrated below.

In some embodiments, system 110 comprises a data analysis module 150. In some embodiments, data analysis module 150 includes identifying and treating systematic errors in sequencing data, as described in connection with data processing module 140.

In some embodiments, system 110 comprises a classification module 160, which analyzes data from a test subject whose status with respect to a medical condition is unknown and subsequently classifies the unknown test subject based on the likelihood of the subject fitting into a particular category. In some embodiments, the one or more parameters include a binomial probability score that is calculated based on logistic regression analysis. As disclosed herein, the binomial probability score may correspond to the likelihood of a subject having a certain medical condition such as cancer. For example, a score of over a predefined threshold may indicate that the subject is more likely to have cancer than not have cancer. In some embodiments, the one or more parameters may include a sequencing data distribution pattern correlating with the presence of cancer. A subject with a pattern resembling the cancer pattern may be diagnosed as having cancer. In some embodiments, a sequencing data distribution pattern may be identified in connection with a specific type of cancer, thus allowing an unknown subject to be classified with further details.

As disclosed herein, network communication module 170 may be used to facilitate communications between a user device, one or more databases, and any other suitable system or device through a wired or wireless network connection. Any communication protocol/device may be used, including without limitation a modem, an Ethernet connection, a network card (wireless or wired), an infrared communication device, a wireless communication device, and/or a chipset (such as a Bluetooth™ device, an 802.11 device, a WiFi device, a WiMax device, cellular communication facilities, etc.), a near-field communication (NFC), a Zigbee communication, a radio frequency (RF) or radio-frequency identification (RFID) communication, a PLC protocol, a 3G/4G/5G/LTE based communication, and/or the like. For example, a user device having a user interface platform for processing/analyzing low coverage sequencing data may communicate with another user device with the same platform, a regular user device without the same platform (e.g., a regular smartphone), a remote server, a physical device of a remote IoT local network, a wearable device, a user device communicably connected to a remote server, and etc.

The functional modules described herein are provided by way of example. It will be understood that different functional modules can be combined to create different utilities. It will also be understood that additional functional modules or sub-modules may be created to implement a certain utility.

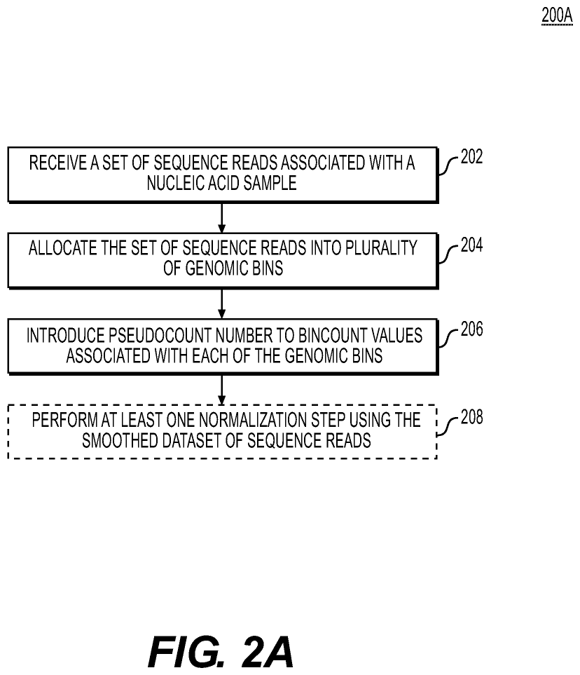

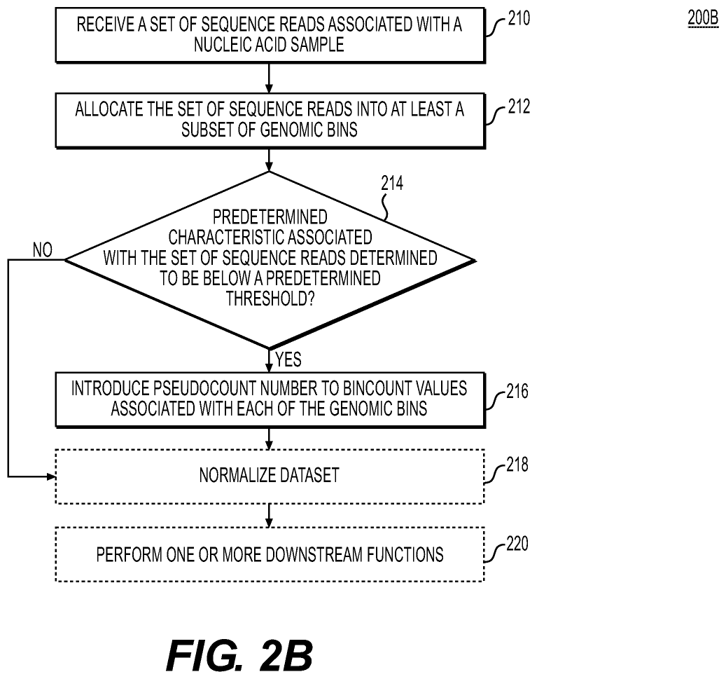

Turning now to FIG. 2A, a flowchart is illustrated of an exemplary method 200A of generating a smoothed dataset for a low coverage set of sequence reads via the utilization of additive smoothing. Exemplary process flows of the method 200A may be performed in accordance with the system 100 above.

At step 202, an embodiment may receive a set of sequence reads associated with a nucleic acid sample. This set of sequence reads may be derived, for example, via implementation of a conventional sequencing process. For instance, a nucleic acid sample (e.g., comprising cell-free DNA (“cfDNA”)) may first be extracted from a subject. The genomic DNA may thereafter be fragmented into shorter pieces that may be further cloned, amplified, purified and sequenced. The quality of the short DNA sequence reads is assessed, ambiguous reads may be removed, and the remaining quality reads may then be assembled into longer contiguous sequences based on comparisons to a reference genome and alignment of overlapping areas. In an embodiment, the sequencing process may be optimized for detection of SCNA data at low coverage depths. It is important to note that although the low coverage sequencing data described herein is derived from off-target data associated with a cfDNA assay, such as targeted DNA methylation, such a situation is not limiting. More particularly, the embodiments described herein may be applicable to virtually any other assay for which the sequencing is performed at a low depth of coverage.

At step 204, an embodiment may allocate the set of sequence reads into a plurality of genomic bins. Each of these genomic bins may represent a small portion of the genome and may contain one or more of sequence reads that are associated therewith. The characteristics of the genomic bins (e.g., size, number, etc.) may be dependent upon a binning algorithm and/or other characteristics of the overarching experiment. To enable the retrieval of genomic information such as that related to SCNAs, binning of short sequence reads is performed in the processing workflow of low-coverage data. An appropriate or optimal bin size and number may be determined that allows for capturing of the depth coverage and trends in the sequenced genome.

The remaining balance of this discussion is described with reference to 100 kb genomic bins. However, such a designation is not limiting and the inventive concepts described herein may be applicable to genomic bins of different size. At high coverage sequencing depths (e.g., 30× coverage, 60× coverage, etc.), the median bincount may be approximately 10,000 sequence reads per 100 kb bin. However, at lower coverage sequencing depths (e.g., <1×-−5× coverage), for which the embodiments herein may pertain to, the median bincount may be relatively low, e.g., 10 reads per 100 kb bin, with some genomic bins containing potentially 0 sequence reads.

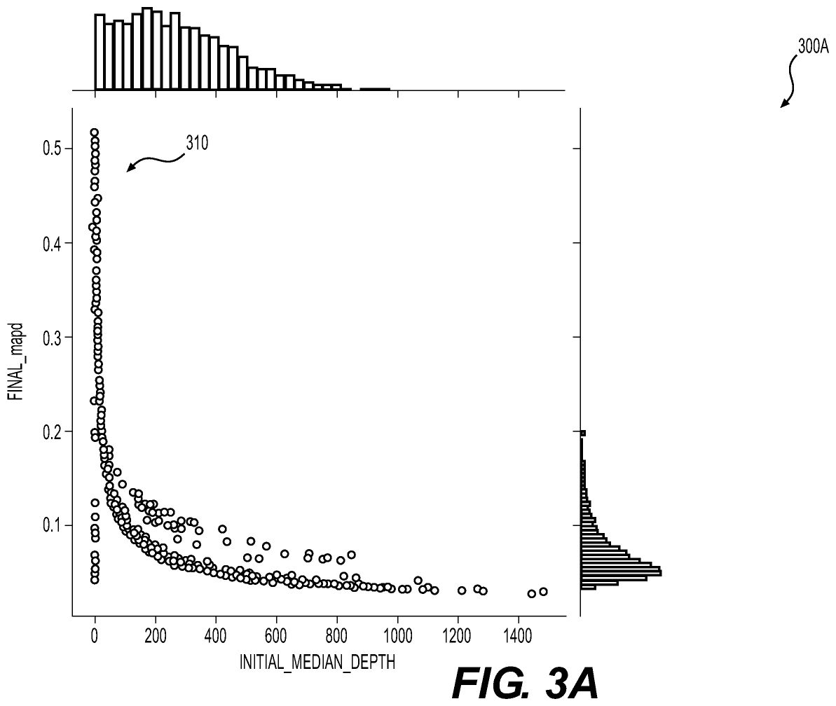

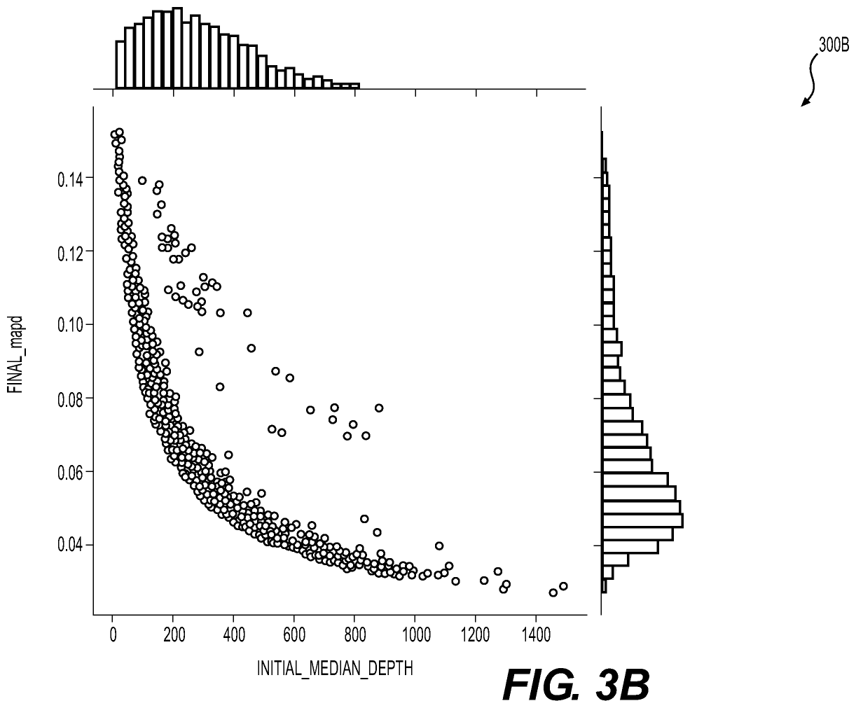

Referring now to FIG. 3A, a graph 300A is illustrated for a set of low coverage whole-genome sequencing datasets for different individuals. The y-axis contains an indication of the median absolute pairwise deviation (“mapd”), which is a measure of noise between adjacent bins after final normalization, and the x-axis provides an indication of the median bin depth. As can be ascertained from examination of the chart, noise 310 is very prevalent at low bin depths (i.e., as indicated by the spike signal). When this normalized data is applied to a disease state classification model (e.g., a cancer classifier, etc.), a “false positive” disease call may be derived. Stated differently, the classification model may output an indication of a registered disease state (e.g., cancer) as a result of the noise 310 when no such disease is actually present.

At step 206, a pseudocount number may be introduced, i.e., added, to the bincount values associated with each of the genomic bins. The addition of the pseudocount number may be part of an additive smoothing technique configured to reduce the noise in the resultant low coverage dataset. For instance, in an exemplary situation, a genomic bin A may contain 2 sequence reads, whereas an adjacent genomic bin B may contain 1 sequence read. Upon adding a pseudocount number of 10 to each of these genomic bins, the relative value of genomic bin A becomes 12 and the relative value of genomic bin B becomes 11, thereby reducing the proportional discrepancy between the values of adjacent genomic bins A and B for a low coverage dataset. In this example, the proportional discrepancy between 12 and 11 is much less than the proportional discrepancy between 2 and 1. Accordingly, when this proportional discrepancy is translated into a graphical format, the resultant effect is a comparatively smoothed version of the dataset.

In an embodiment, the decision to introduce a pseudocount number to the genomic bins may be made manually by an individual (e.g., via interaction with a computing device via I/O Module 120). For instance, an individual may examine the generated graph (e.g., as illustrated in FIG. 3A) and determine that the resultant data is too noisy and may cause downstream issues, e.g., by identifying that the median depth of the sample is too low. In another embodiment, the decision to introduce the pseudocount number to the genomic bins may be automated. For example, the system 100 may be configured to add a pseudocount number to each of the genomic bins in every sequencing sample (i.e., regardless of coverage depth). Alternatively, the system 100 may be configured to only add a pseudocount number to those sets of sequence reads for which the depth of coverage is below a predetermined threshold. This predetermined threshold may be adjustable by a user. Alternatively, the system 100 may be configured to only add a pseudocount number to those sets of sequence reads for which a predetermined characteristic associated with the set of sequence reads, e.g., a median or mean bincount value, is below a predetermined threshold.

In an embodiment, the value of the pseudocount number may be selected based on the unique characteristics of the dataset being analyzed (e.g., bin size, sequencing depth, etc.). For instance, an optimal pseudocount number (i.e., a number that optimizes classification performance and/or that is configured to best reduce the noise associated with low bin depth data without significantly skewing the proportional discrepancy between adjacent bins at normal or high bins depths) for a particular dataset may be selected by trying a plurality of different positive numbers (e.g., whole numbers, decimals, etc.). Once an optimal pseudocount number is selected, that pseudocount number may be applied uniformly throughout the dataset. Alternatively, the value of the pseudocount number may be dynamically determined by the system. This determination may be based on one or more factors, e.g., a bincount value, a mapd value, a coverage value of the sequencing set, and/or a cross validation metric associated with data from other samples. In other embodiments, a standard, predetermined pseudocount number may be automatically added to each dataset. It is important to note that although this disclosure is explained with reference to a pseudocount number of 10, such a designation is not limiting and any other suitable positive integer may be designated as the pseudocount number. A suitable pseudocount number may be, for example, a number ranging from 5 to 100 or 5 to 500. A suitable pseudocount number may be selected such that the pseudocount number is large enough to smooth proportional discrepancies between adjacent bins with low coverage, but not so large so as to smooth meaningful discrepancies between adjacent bins with normal or high sequence coverage that may be indicative of, e.g., a disease state.

The addition of the pseudocount number to the genomic bin values in step 206 may produce a smoothed dataset. Once the smoothed dataset is produced, an optional normalization step 208 may be performed on the smoothed dataset. Normalization step 208 may itself include the application of one or more than one normalization process. As a practical implementation of the foregoing concepts, and with reference to FIG. 3B, a graph 300B is illustrated in which a pseudocount number (in this case, 10) has been added to the genomic bin values associated with the set of sequence reads utilized to generate graph 300A in FIG. 3A. Upon comparison of graph 300B to graph 300A, it can be seen that the addition of the pseudocounts has served to smooth out the noise from graph 300A because the proportional discrepancies between adjacent bins (i.e., the mapd values) with low coverage become less extreme.

Turning now to FIG. 2B, a flowchart is illustrated of an exemplary method 200B of generating a smoothed dataset for a low coverage set of sequence reads via the utilization of additive smoothing. Exemplary process flows of the method 200B may be performed in accordance with the system 100 above.

At step 210, an embodiment may receive a set of sequence reads associated with a nucleic acid sample (as previously described above with reference to step 202 in FIG. 2A) and may thereafter allocate, at step 212, the set of sequence reads into a plurality of genomic bins (as previously described above with reference to step 204 in FIG. 2A).

At step 214, an embodiment may dynamically determine whether a predetermined characteristic associated with the set of sequence reads is below a predetermined threshold. In an embodiment, the predetermined characteristic associated with the set of sequence reads may be one or more of: a sequencing coverage depth, a median bin count value, a mean bin count value, the presence of a subset of one or more bins with no sequence coverage, the presence of a subset of one or more bins with sequence coverage below a predetermined threshold amount, and/or another metric associated with the set of sequence reads. In an embodiment, the predetermined threshold for each type of predetermined characteristic may be manually set by a user or, alternatively, may be dynamically established by computer system 110. More particularly, with respect to the latter, computer system 110 may leverage database 130 and/or data processing module 140 to identify a value for the predetermined threshold for each type of predetermined characteristic. As a non-limiting example of the foregoing, computer system 110 may access and analyze prior experiment data, available crowdsourced data, etc., to determine that 10× was the average minimum coverage depth that produced reliable results. From this determination, computer system 110 may thereafter dynamically establish 10× coverage as the predetermined threshold (i.e., which may represent the delineation between a low sequencing coverage depth and a normal or high sequencing coverage depth) when an embodiment relies on coverage depth as the predetermined characteristic.

Responsive to determining, at step 214, that the predetermined characteristic is determined to be above the predetermined threshold, an embodiment may, at step 218, optionally proceed to normalize the dataset of original sequence reads. Conversely, responsive to determining, at step 214, that the predetermined characteristic is determined to be below the predetermined threshold, an embodiment may, at step 216, dynamically introduce a pseudocount number to bincount values associated with each of the genomic bins, as previously described with reference to step 206 in FIG. 2A. Thereafter, an embodiment may, at step 218, optionally proceed to normalize the pseudo-count modified set of sequence reads, similar to optional step 208 in FIG. 2A. For instance, in methods 200A and 200B, an initial, within-sample normalization step may be conducted on the pseudocount-modified set of sequence reads. This first normalization step may be based on, for example, a median bincount value. Subsequently, one or more other normalization steps may occur (e.g., cross-sample normalization with sets of sequence reads from other “baseline” samples, etc.).

The normalized data then may, at step 220, optionally be utilized in the performance of one or more downstream functions. For example, the normalized data may be applied as an input vector to a disease state classification model, such as a cancer classifier. The application of the pseudocount-modified dataset may improve the performance of the disease state classification model by providing results with less noise than when an original, low coverage dataset or a subsampled dataset is applied to the classification model. For instance, the sensitivity, specificity, number of cancers detected, and number of joint calls (i.e., the number of times the SCNA assay confirmed a cancer call by the targeted DNA methylation assay) all exhibited improved metrics for the pseudocount-modified dataset over the original, low coverage dataset as well as the subsampled dataset. The application of a pseudocount may or may not affect the normalization step(s) performed. For example, the smoothing of data using the pseudocount may cause a different normalization method, or a different set of normalization methods, to be used to standardize the data.

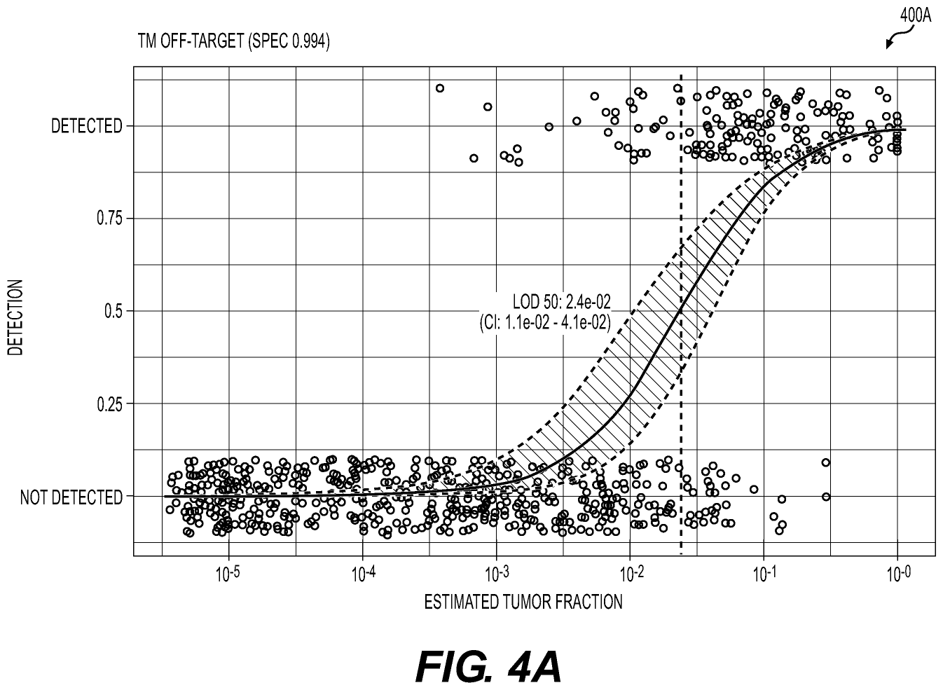

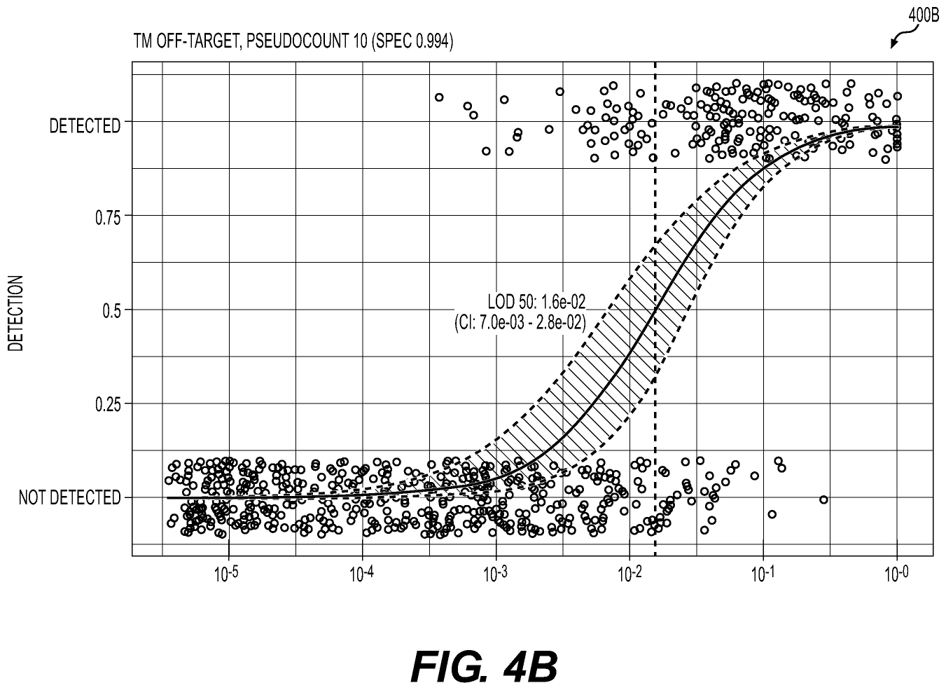

Referring now to FIGS. 4A-B, results from the incorporation of the pseudocounts to the genomic bin values for various sets of sequence reads may lower the limit of detection (LoD) of a cancer classifier (i.e., the ability of a classifier to detect tumor DNA at lower concentrations in a sample). More particularly, turning now to FIG. 4A, graph 400A is presented in which the LoD of the off-target targeted DNA methylation data based on the original, unaltered sequence reads is determined to be 2.4e⁻². This LoD is higher than the off-target targeted DNA methylation data based on the pseudocount-modified sequence reads, as illustrated in graph 400B in FIG. 4B, which is determined to be 1.6e⁻². Accordingly, the analytical sensitivity of a cancer classifier utilizing pseudocount-modified sequence reads may be effectively increased (i.e., as a result of an improved signal-to-noise ratio), thereby enabling the pseudocount-based cancer classifier to identify tumor-derived DNA molecules at lower fractional amounts. The dataset in FIGS. 4A-B is based on the same exemplary dataset used in FIGS. 3A-B. A comparison of FIGS. 4A-B suggests that “reasonable” cancer detections are gained, i.e. that the new detections aren't at an unreasonably low tumor fraction that would indicate that the new detections are artifacts rather than cancer.

In some embodiments, the methods, systems, and/or classifier of the present disclosure can be used to detect the presence (or absence) of cancer, monitor cancer progression or recurrence, monitor therapeutic response or effectiveness, determine a presence or monitor minimum residual disease (MRD), or any combination thereof. In some embodiments, the systems and/or classifier may be used to identify the tissue or origin for a cancer. For instance, the systems and/or classifiers may be used to identify a cancer as of any of the following cancer types: head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer. In some embodiments, a test report can be generated to provide a patient with their test results, including, for example, a probability score that the patient has a disease state (e.g., cancer), a type of disease (e.g., a type of cancer), and/or a disease tissue of origin (e.g., a cancer tissue of origin). In some embodiments, the methods and/or classifier of the present disclosure are used to detect the presence or absence of cancer in a subject suspected of having cancer. According to aspects of the disclosure, the methods and systems of the present disclosure can be trained to detect or classify multiple cancer indications. For example, the methods, systems, and classifiers of the present disclosure can be used to detect the presence of one or more, two or more, three or more, five or more, or ten or more different types of cancer. In some embodiments, the cancer is one or more of head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer.

The smoothed dataset may be generated for sequence data obtained from a cancer patient over any desired set of time points and analyzed in accordance with the methods of the disclosure to monitor a cancer state in the patient. In some embodiments, a first time point is before a cancer treatment (e.g., before a resection surgery or a therapeutic intervention), and the second time point is after a cancer treatment (e.g., after a resection surgery or therapeutic intervention), and the method is utilized to monitor the effectiveness of the treatment. In other embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment. In still other embodiments, smoothed dataset may be generated for sequence data obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, smoothed datasets can be generated for sequence data obtained from the patient at least once every 3 months, at least once every 6 months, at least once a year, at least once every 2 years, at least once every 3 years, at least once every 4 years, or at least once every 5 years.

In still another embodiment, information obtained from any method described herein can be used to make or influence a clinical decision (e.g., diagnosis of cancer, treatment selection, assessment of treatment effectiveness, etc.). For example, in one embodiment, a physician can prescribe an appropriate treatment (e.g., a resection surgery, radiation therapy, chemotherapy, and/or immunotherapy) based on the utilization of the smoothed dataset in a classification process. In some embodiments, information such as classification based on the smoothed dataset can be provided as a readout to a physician or subject. In some embodiments, classification based on the smoothed dataset can indicate the effectiveness of a cancer treatment.

In some embodiments, the treatment is one or more cancer therapeutic agents selected from the group including a chemotherapy agent, a targeted cancer therapy agent, a differentiating therapy agent, a hormone therapy agent, and an immunotherapy agent. For example, the treatment can be one or more chemotherapy agents selected from the group including alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group including signal transduction inhibitors (e.g., tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group including anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). The appropriate cancer therapeutic agent can be selected based on characteristics such as the type of tumor, cancer stage, previous exposure to cancer treatment or therapeutic agent, and other characteristics of the cancer.

In addition to a standard desktop, or server, it is fully within the scope of this disclosure that any computer system capable of the required storage and processing demands would be suitable for practicing the embodiments of the present disclosure. This may include tablet devices, smart phones, pin pad devices, and any other computer devices, whether mobile or even distributed on a network (i.e., cloud based).

Unless specifically stated otherwise, as apparent from the following discussions, it is appreciated that throughout the specification discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer,” a “computing machine,” a “computing platform,” a “computing device,” or a “server” may include one or more processors.

In accordance with various implementations of the present disclosure, the methods described herein may be implemented by software programs executable by a computer system. Further, in an exemplary, non-limited implementation, implementations may include distributed processing, component/object distributed processing, and parallel payment. Alternatively, virtual computer system processing may be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP, etc.) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments, various features of the embodiments are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that a claimed embodiment requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment.

Furthermore, while some embodiments described herein include some but not other features included in other embodiments, combinations of features of different embodiments are meant to be within the scope of the present disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the function.

In the description provided herein, numerous specific details are set forth. However, it is understood that embodiments of the present disclosure may be practiced without these specific details. In other instances, well-known methods, structures, and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the present disclosure, those skilled in the art will recognize that other and further modifications may be made thereto without departing from the spirit of the present disclosure, and it is intended to claim all such changes and modifications as falling within the scope of the present disclosure. For example, any formulas given above are merely representative of procedures that may be used. Functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

What is claimed is:

1. A method of reducing noise for the analysis of low coverage sequencing data from a nucleic acid sample using a system, the method comprising:
receiving, at an input component of the system, a set of sequence reads associated with the nucleic acid sample;
allocating, using a processor component of the system, the set of sequence reads into a plurality of genomic bins; and
introducing, subsequent to the allocating, a pseudocount number to bincount values to produce a smoothed dataset, wherein each of the bincount values is associated with one of the plurality of genomic bins.

2. The method of claim 1, wherein the low coverage sequencing data is derived at least partially from off-target data associated with a cell free DNA (cfDNA) assay.

3. The method of claim 2, wherein the cfDNA assay is a targeted DNA methylation assay, and wherein the low coverage sequencing data corresponds to somatic copy number alteration (SCNA) sequencing data.

4. The method of claim 1, wherein the introducing comprises automatically introducing the pseudocount number responsive to identifying that a predetermined characteristic associated with the set of sequence reads is below a predetermined threshold.

5. The method of claim 1, wherein the introducing comprises automatically determining the pseudocount number based on at least one factor.

6. The method of claim 5, wherein the at least one factor is selected from the group consisting of: a bincount value metric, a median absolute pairwise deviation (mapd) metric, and a cross validation metric.

7. The method of claim 1, wherein the pseudocount number is 10.

8. The method of claim 1, further comprising performing, using the smoothed dataset, at least one normalization step on the set of sequence reads for the nucleic acid sample.

9. The method of claim 8, further comprising:
applying, to a classification model associated with a disease state, the normalized set of sequence reads; and
determining, from results derived from the applying, whether an indication of the disease state is detected from the normalized set of sequence reads.

10. The method of claim 9, wherein the disease state is cancer.

11. A system for reducing noise for the analysis of low coverage sequencing data from a nucleic acid sample, the system comprising:
a database; and
at least one processing component configured to perform operations including:
receiving a set of sequence reads associated with the nucleic acid sample;
allocating the set of sequence reads into a plurality of genomic bins; and
introducing, subsequent to the allocating, a pseudocount number to bincount values to produce a smoothed dataset, wherein each of the bincount values is associated with one of the plurality of genomic bins.

12. The system of claim 11, wherein the low coverage sequencing data is at least partially derived from off-target data associated with a cell free DNA (cfDNA) assay.

13. The system of claim 12, wherein the cfDNA assay is a targeted DNA methylation assay, and wherein the low coverage sequencing data corresponds to somatic copy number alteration (SCNA) sequencing data.

14. The system of claim 11, wherein the operations to introduce further comprise:
automatically introducing the pseudocount number responsive to identifying that a predetermined characteristic associated with the set of sequence reads is below a predetermined threshold.

15. The system of claim 11, wherein the operations to introduce further comprise:
automatically determining the pseudocount number based on at least one factor.

16. The system of claim 15, wherein the at least one factor is selected from the group consisting of: a bincount value metric, a median absolute pairwise deviation (mapd) metric, and a cross validation metric.

17. The system of claim 11, wherein the operations further comprise:
performing, using the smoothed dataset, at least one normalization step on the set of sequence reads for the nucleic acid sample.

18. The system of claim 17, wherein the operations further comprise:
applying, to a classification model associated with a disease state, the normalized set of sequence reads; and
determining, from results derived from the applying, whether an indication of the disease state is detected from the normalized set of sequence reads.

19. The system of claim 18, wherein the disease state is cancer.

20. A non-transitory computer-readable medium storing instructions for reducing noise for the analysis of low coverage sequencing data from a nucleic acid sample, the instructions, when executed by one or more processors, causing the one or more processors to perform operations comprising:
receiving a set of sequence reads associated with the nucleic acid sample;
allocating the set of sequence reads into a plurality of genomic bins; and
introducing, subsequent to the allocating, a pseudocount number to bincount values to produce a smoothed dataset, wherein each of the bincount values is associated with one of the plurality of genomic bins.
